SEQUENCE LISTING (5 sequences)

SEQ ID NO: 1 - protein, 348 aa, Fusarium heterosporum
MLLLSLLSAVTLAVASPVALEEYANSLEDRAVGVTSTDFTNFKFYIQHGAAAYCNSGTAAGAKITCSNNGCPTIESNGVTVVASFTGSKTGIGGYVSTDSSRKEIVVAIRGSSNIRNWLTNLDFDQSDCSLVSGCGVHSGFQNAWAEISAQASAAVAKARKANPSFKVVATGHSLGGAVATLSAANLRAAGTPVDIYTYGAPRVGNAALSAFISNQAGGEFRVTHDKDPVPRLPPLIFGYRHTTPEYWLSGGGGDKVDYAISDVKVCEGAANLMCNGGTLGLDIDAHLHYFQATDACNAGGFSWRRYRSAKRESIDMRATMTDAQLEAKLNSYVAMDQEYVKTHQNRT

SEQ ID NO: 2 - protein, 346 aa, Fusarium sp
MLLLPLLSAITLAVASPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKRATMTDAELEKKLNSYVQMDKEYVKNNQARS

SEQ ID NO: 3 - protein, 346 aa, Fusarium decemcellulare
MLFLSLLSAATLAVASPLADYVRALDDRAVSVSSQDYNNFKFYIQHGAAAYCNSETAAGQKITCGENSCPTVQSNGATVVASFVGSGTGIGGYVATDPTRKEIVLSFRGSSNIRNWLANLDFGQSDCTLTSNCGVHTGFQNAWNEVASKAKAAISSALKSNPTYKVIATGHSLGGAVATIGAANLRAGGTPVDIYTYGSPRVGNSELAAFISNQAGGEYRVTHGRDPVPRLPPLVFGYRHTSPEYWLSGGASDQNDYAITSIKTCEGGANLACNGGTLGLDIVAHLHYFQGTSDCNPGGIEWKRDTPAKRNKLVKRAEMTDAELEKKLNNYVQMDKEYVANHKDRS

SEQ ID NO: 4 - protein, 332 aa, Dactylonectria estremocensis
MLVLSLLSSVGVAAAASIGNYARILEDRAVTVTSANLANFKFYAQHSAAAYCNYGAKAGAKISCGGYCPTVESDGAVIVASFGGSATGIAGYVSTDATRKEIVISIRGSSNIRNWITNLDFGQDSCSSLVSGCGVHSGFQNAWDEIAAAAKAAVVSARTVNPSYVVVATGHSLGGAVATLAAAYLRKAGIPMDLYTFGSPRVGDGDFANFVTSQAGAEYRVTHEADPVPRLPPIIFGYRHTSPEYWLTGGSSDTTTYSVSSIKVCEGNANIGCNAGTLGLDIIAHLNYLQDISGCAAIGISWRRDELTDAELEAKINQYAKLDVEYVSYQPS

SEQ ID NO: 5 - protein, 177 aa, Metarhizium guizhouense
MKLNTTLLALATTALAAPASDVTTPKRQDINTVTDQLLFSSTLSQFEARRNAKEPPSLDWSSDGCTSSPDNPFGFPFLPACHRHDFGYQNYRIQKRFTKAAKAKIDSNFKTDLYYQCRSVSAKDACNGLADVYYEAVKEFGGGDATKRDRSDYDRAVAAYNAAVKEAQEQGLLPILD

(19) United States
(12) Patent Application Publication
(10) Pub. No.: US 2026/0305624 A1
(43) Pub. Date: Oct. 8, 2026

(54) ENZYMATIC MODIFICATION OF GALACTOLIPIDS IN FOOD

(71) Applicant: INTERNATIONAL N&H DENMARK APS, Kongens Lyngby (DK)

(72) Inventors: Svend Haaning, Galten (DK); Tina Lillan Jøergensen, Kongens Lyngby (DK); Lene Kragh, Aarhus (DK); Lone Broend Miller, Aarhus C (DK); Jorn Borch Soe, Tilst (DK); Kefeng Ni, Shanghai (CN); Yuepeng Shang, Shanghai (CN); Xingxiang Xi, Shanghai (CN); Helong Hao, Shanghai (CN); Jens Frisbaek Sørensen, Aarhus N (DK)

(21) Appl. No.: 19/717,108

(22) Filed: Jun. 23, 2026

Related U.S. Application Data
(63) Continuation of application No. 18/711,247, filed on Nov. 17, 2022.

(30) Foreign Application Priority Data
Nov. 17, 2021 (WO) PCT/CN2021/131128

(51) Int. Cl. A21D 8/04; A21D 10/00; A23K 20/189; A23L 15/00; C12N 9/18

(52) U.S. Cl. CPC A21D 8/042; A21D 10/002; A23K 20/189; A23L 15/25; C12N 9/18; C12Y 301/01026

(57) ABSTRACT

The present relates to lipases having galactolipase activity and no/low lyso-phospholipase activity. More particularly, the present invention relates to the use of such lipases for generation of emulsifying components from endogenous flour lipids.

### CROSS-REFERENCE TO RELATED APPLICATIONS

This application is a continuation of U.S. application Ser. No. 18/711,247, filed May 17, 2024, which is a U.S. National Stage Application under 35 U.S.C. § 371 of International Application No. PCT/US2022/080062, filed Nov. 17, 2022, which claims priority to International Application No. PCT/CN2021/131128, filed Nov. 17, 2021, all of which are hereby incorporated by reference in their entireties.

### INCORPORATION BY REFERENCE OF THE SEQUENCE LISTING

The present application is being filed with a Sequence Listing in electronic format. The Sequence Listing is provided as a file entitled NB42020USPCT2_SequenceListing.xml, created on May 10, 2024, which is 7,168 bytes in size. The information in the electronic format of the Sequence Listing is incorporated by reference in its entirety.

### TECHNICAL FIELD

The present invention relates to lipases and their use in the manufacture of food. The present invention further relates to methods of making dough and baked products using such lipases.

### BACKGROUND

The use of lipases to provide improved bread dough is well known. For example, in EP0585988 it is shown that the addition of a lipase to dough provided an anti-staling effect in bread baked therefrom. WO94/04035 teaches that an improved softness can be obtained by adding a lipase to dough. It has also been shown that exogenous lipases can modify bread volume.

While lipases have been described for their positive properties in the preparation of dough and baked products, the performance of prior art lipases has many drawbacks because prior art lipases have generally had multiple activities, reducing or eliminating the potential beneficial effect of the lipase. Therefore, there is a need in some food applications, particularly in baking, for improved lipases having higher specificity.

### SUMMARY OF THE INVENTION

In accordance with an aspect of the present invention, an isolated polypeptide is presented constituting a lipase having galactolipase activity of at least 100 DGDG-U per mg of the lipase and having a ratio of LPC-U/DGDG-U below 0.02. Optionally, the lipase has galactolipase activity of at least 150, 200, 250 or 300 DGDG-U per mg.

Optionally, the lipase has a ratio of LPC-U/DGDG-U below 0.01, 0.009, 0.007, 0.005, 0.003 or 0.001.

Optionally, the lipase is an engineered lipase.

In another aspect of the present invention, a nucleic acid sequence is presented encoding the isolated polypeptide as described above.

In another aspect of the present invention, a recombinant expression vector is presented having the polynucleotide as described above.

In another aspect of the present invention, a host cell is presented having the recombinant expression vector as described above.

In another aspect of the present invention, a method is presented of making a dough, the method having the step of admixing a dough component selected from the group consisting of flour, salt, water, sugar, fat, lecithin, oil and yeast with an isolated polypeptide as described above.

Optionally, the method includes adding at least one additional enzyme useful for improving dough and/or a baked product made therefrom. Optionally, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Optionally, the additional enzyme is an amylase which is optionally an exoamylase. Optionally, the exoamylase is a maltogenic amylase. Optionally, the exoamylase is a non-maltogenic amylase. Optionally, the non-maltogenic amylase hydrolyses starch by cleaving off one or more linear malto-oligosaccharides, predominantly comprising from four to eight D-glucopyranosyl units, from the non-reducing ends of the side chains of amylopectin. Optionally, the additional enzyme is a phospholipase. Optionally, the phospholipase comprises SEQ ID NO: 5 or an active fragment thereof. Optionally, the active fragment is a mature polypeptide.

Optionally, the method includes adding an emulsifier. Optionally, the emulsifier is a phospholipid emulsifier. Optionally, the phospholipid emulsifier is lecithin or lyso-lecithin. Optionally, the emulsifier is a non-phospholipid emulsifier. Optionally, non-phospholipid emulsifier is DATEM, SSL, a monoglyceride or a diglyceride.

In another aspect of the present invention, a pre-mix for baking is presented having flour and an isolated polypeptide as described above.

Optionally, the pre-mix includes adding at least one additional enzyme useful for improving dough and/or a baked product made therefrom. Optionally, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Optionally, the additional enzyme is an amylase which is optionally an exoamylase. Optionally, the exoamylase is a maltogenic amylase. Optionally, the exoamylase is a non-maltogenic amylase. Optionally, the non-maltogenic amylase hydrolyses starch by cleaving off one or more linear malto-oligosaccharides, predominantly comprising from four to eight D-glucopyranosyl units, from the non-reducing ends of the side chains of amylopectin. Optionally, the additional enzyme is a phospholipase. Optionally, the phospholipase comprises SEQ ID NO: 5 or an active fragment thereof. Optionally, the active fragment is a mature polypeptide.

In another aspect of the present invention, a method of creating a lyso-galactolipid in a lipid containing food matrix is presented having the step of adding to the lipid containing food matrix an isolated polypeptide as described above. Optionally, the lipid containing food matrix is selected from the group consisting of eggs and food products containing eggs, dough for sweet bakery goods, processed meat, milk based products, and vegetable oil.

In another aspect of the present invention, a method of creating a lyso-galactolipid in a lipid containing animal feed matrix or a grain based matrix is presented having the step of adding to the lipid containing to the lipid containing animal feed matrix or the grain based matrix an isolated polypeptide as described above.

In another aspect of the present invention, a dough is presented having an isolated polypeptide as described above. Optionally, the dough has improved dough extensibility and/or stability. Optionally, the dough has at least one additional enzyme useful for improving dough and/or a baked product made therefrom. Optionally, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Optionally, the additional enzyme is an amylase which is optionally an exoamylase. Optionally, the exoamylase is a maltogenic amylase. Optionally, the exoamylase is a non-maltogenic amylase. Optionally, the non-maltogenic amylase hydrolyses starch by cleaving off one or more linear malto-oligosaccharides, predominantly comprising from four to eight D-glucopyranosyl units, from the non-reducing ends of the side chains of amylopectin. Optionally, the additional enzyme is a phospholipase. Optionally, the phospholipase comprises SEQ ID NO: 5 or an active fragment thereof. Optionally, the active fragment is a mature polypeptide.

In another aspect of the present invention, a method of preparing a baked product is presented having the step of baking a dough as described above.

In another aspect of the present invention, a baked product is presented prepared according to the method as described above. Optionally, the baked product has at least one improved property selected from the group consisting of improved crumb pore size, improved uniformity of gas bubbles, no separation between crust and crumb, increased volume, increased crust crispiness and improved oven spring.

In another aspect of the present invention, an isolated polypeptide is presented constituting a lipase having at least 75, 80, 85, 90, 95, 98, 99 or 100% sequence identity to SEQ ID NO:3 or SEQ ID NO:4 or a lipase active fragment thereof. Optionally, the lipase active fragment is a mature polypeptide.

### BRIEF DESCRIPTION OF THE BIOLOGICAL SEQUENCES

SEQ ID NO: 1 sets forth the full length amino acid sequence of a lipase used in the commercial product Powerbake 4080.

SEQ ID NO: 2 sets forth the full length amino acid sequence of a lipase used in the commercial product Lipopan F.

SEQ ID NO:3 sets forth the full length amino acid sequence of CRC26230.

SEQ ID NO:4 sets forth the full length amino acid sequence of CRC26190.

SEQ ID NO:5 sets forth the full length amino acid sequence of the phospholipase (see also WO2019/121585 incorporated here by reference).

### BRIEF DESCRIPTION OF THE DRAWINGS

FIG. 1 depicts crusty roll volumes presented relative to volume of crusty roll with ‘SEQ ID NO:5’.

### DETAILED DESCRIPTION OF THE INVENTION

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Abbreviations

PL—phospholipid
LPL—lyso-phospholipid
GL—galactolipid
LGL—lyso-galactolipid
DGDG—di-galactosyl-di-glyceride
DGMG—di-galactosyl-mono-glyceride
MGDG—mono-galactosyl-di-glyceride
MGMG—mono-galactosyl-mono-glyceride
PC—phosphatidylcholine
LPC—lyso-phosphatidylcholine\
PLA—phospholipase A
FFA—free fatty acid
PC-U—phospholipase activity
LPC-U—lyso-phospholipase activity
DGDG-U—galactolipase activity

### Definitions

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a galactolipase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptide, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 80% pure, at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and there may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ. Very stringent hybridization conditions involve 68° C. and 0.1×SSC.

A “synthetic” molecule is produced by in vitro chemical or enzymatic synthesis rather than by an organism.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a phospholipase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein. Alternatively, specific activity can refer to the number of moles of product generated by an enzyme or enzyme preparation per unit of time under specific conditions.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty: | OFF |

Deletions are counted as non-identical residues, compared to a reference sequence. Deletions occurring at either terminus are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

As used herein, the singular forms “a”, “an”, and “the” include both singular and plural referents unless the context clearly dictates otherwise.

The terms “comprising”, “comprises” and “comprised of as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any >3, >4, >5, >6 or >7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the term “lipase” refers to subset of Carboxylic Ester Hydrolases (EC 3.1.1) which catalyze the hydrolysis of ester bounds in lipids including triglycerides, phospholipids and galactolipids. It is known to those of skill in the art that lipases have a broad substrate range. Thus, for example, a phospholipase may also have activity on substrates other than phospholipids. For example, what is described as a phospholipase may be active on galactolipids. In this regard, a galactolipase may act on phospholipids. In general, the predominant activity of a lipase will be used to assign it to a class, e.g., galactolipases. Hence, a lipase of the instant invention may have activity towards different lipid moieties.

As used herein, the term “phospholipase” refers to an enzyme that hydrolyses phospholipids into fatty acids (saturated and/or unsaturated), lyso-phospholipids, diacylgycerols, choline phosphate and phophatidates, depending on the site of hydrolysis. Phospholipases are further classified into types A, B, C and D.

As used herein, the term “phospholipase A” refers to enzymes that catalyze the hydrolysis of the ester bond of the fatty acid components of phospholipids. There are two different types of phospholipase A activity that can be distinguished. Phospholipase A1, as defined in enzyme entry EC 3.1.1.32, and phospholipase A2, as defined in enzyme entry EC 3.1.1.4, catalyze the deacylation of one fatty acyl group in the sn1 and sn2 positions, respectively, from a di-acyl-glycerophospholipid to produce lyso-phospholipid.

Another phospholipase is a “lyso-phospholipase” which catalyzes the hydrolysis of the fatty acyl group in the lyso-phospholipid.

As used herein, the term “galactolipase” refers to an enzyme that hydrolyses galactolipids into fatty acids (saturated and/or unsaturated), lyso-galactolipids and galactosylglycerols. Galactolipases catalyze the hydrolysis of galactolipids including di-galactosyl-di-glyceride (DGDG) and mono-galactosyl-di-glyceride (MGDG).

Another galactolipase is a “lyso-galactolipase” which catalyzes the hydrolysis of the fatty acyl group in the lyso-galactolipid.

As used herein, the phrase “a lyso-phospholipase/phospholipase activity ratio” or LPL/PL activity ratio′ means (LPC-U/mg protein)/(PC-U/mg protein) as set forth more fully below.

As used herein, the phrase “a lyso-phospholipase/galactolipase activity ratio” or LPL/GL activity ratio′ means (LPC-U/mg protein)/(DGDG-U/mg protein) as set forth more fully below.

As used herein, the phrase “engineered lipase” means a variant lipase obtained by altering a wildtype lipase through the use of protein engineering tools, known by a skilled person in the art. Many of the technologies are described in Protein Engineering Handbook, Vol I and II. WILEY-VCH Verlag GmbH & Co. KGaA. Ed. Stefan Lutz and Uwe T. Bornscheuer.

As used herein, the terms “phospholipids” or “phospholipid” or “PL” share the common features of fatty acids esterified to the 1 and 2 positions of the glycerol backbone with the phosphate group esterified to the 3 position.

As used herein, the terms “lyso-phospholipids”, “lyso-phospholipid” and “LPL” refer to a phospholipid containing one fatty acid esterified to the 1 or 2 position of the glycerol backbone with a phosphate group esterified to the 3 position.

As used herein, the term “galactolipids” or “galactolipid” or “GL” refer to esters of glycerol, fatty acids and sugar groups. The most common galactolipids are (but not limited to) mono-galactosyl-di-glyceride (MGDG) and di-galactosyl-di-glyceride (DGDG). These galactolipids share the common features of fatty acids (saturated and/or unsaturated) esterified to the 1 and 2 positions of the glycerol backbone with a sugar group linked to the 3 position.

As used herein, the terms “lyso-galactolipids”, “lyso-galactolipid”, and “LGL” refer to a galactolipid, containing one fatty acid esterified to the 1 or 2 position of the glycerol moiety and a sugar group linked to the 3 position.

As used herein, the phrase “lipid containing food matrix” refers to any food matrix that contains amounts of lipids.

Other definitions are set forth below.

### Additional Mutations

In some embodiments, the lipases of the present invention include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature. In some cases, the performance benefit may only be realized in the application e.g. bread making.

Furthermore, the lipases of the present invention may include any number of conservative amino acid substitutions. Exemplary conservative amino acid substitutions are listed in Table 1 below.

TABLE 1
Conservative Amino Acid Changes

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, |
| | | D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, D- |
| | | Met, Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4, or 5-phenylproline, cis-3,4, |
| | | or 5-phenylproline |
| Proline | P | D-Pro, L-I-thioazolidine-4-carboxylic acid, D-or |
| | | L-1-oxazolidine-4-carboxylic acid |
| Serine | S | D-Ser, Thr, D-Thr, allo-Thr, Met, D-Met, Met(O), |
| | | D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Those of skill in the art will appreciate that some of the above-mentioned conservative mutations can be produced by genetic manipulation, while others are produced by introducing synthetic amino acids into a polypeptide by genetic or other means.

The lipases of the present invention may be “precursor,” “immature,” or “full-length,” in which case they include a signal sequence, or “mature,” in which case they lack a signal sequence and may be further truncated at the N- and/or C-terminus by proteolytic and/or non-proteolytic processing. In general, the mature forms of the polypeptides are generally the most useful. Unless otherwise noted, the amino acid residue numbering used herein refers to the mature forms of the respective lipase polypeptides. The present lipase polypeptides may also be truncated to remove the N or C-termini, so long as the resulting polypeptides retain lipase activity. In addition, lipase enzymes may be active fragments derived from a longer amino acid sequence. Active fragments are characterized by retaining some or all of the activity of the full-length enzyme but have deletions from the N-terminus, from the C-terminus or internally or combinations thereof.

The lipases of the present invention may be “chimeric” or “hybrid” polypeptides, in that they include at least a portion of a first lipase polypeptide, and at least a portion of a second lipase polypeptide. The lipases of the present invention may further include heterologous signal sequence, an epitope to allow tracking or purification, or the like. Exemplary heterologous signal sequences are from B. licheniformis amylase (LAT), B. subtilis (AmyE or AprE), T. reesei (AFP) and Streptomyces (CelA).

### DESCRIPTION OF THE PREFERRED EMBODIMENTS

In accordance with an aspect of the present invention, an isolated polypeptide is presented constituting a lipase having galactolipase activity of at least 100 DGDG-U per mg of the lipase and having a ratio of LPC-U/DGDG-U below 0.02. Preferably, the lipase has galactolipase activity of at least 150, 200, 250 or 300 DGDG-U per mg.

Without being bound by theory, the novel lipases of the present invention have a narrower substrate specificity than prior art lipases. The novel lipases of the instant invention produce less free fatty acid as compared with prior art baking lipases while maintaining very good baking performance. Controlled formation of free fatty acid on a lower level compared with prior art baking lipase is very important because free fatty acids are not preferred in food systems because they might give rise to oxidation and off-flavor formation

Preferably, the lipase has a ratio of LPC-U/DGDG-U below 0.01, 0.009, 0.007, 0.005, 0.003 or 0.001.

Preferably, the lipase is an engineered lipase.

In another aspect of the present invention, a nucleic acid sequence is presented encoding the isolated polypeptide as described above.

In another aspect of the present invention, a recombinant expression vector is presented having the polynucleotide as described above.

In another aspect of the present invention, a host cell is presented having the recombinant expression vector as described above.

In another aspect of the present invention, a method is presented of making a dough, the method having the step of admixing a dough component selected from the group consisting of flour, salt, water, sugar, fat, lecithin, oil and yeast with an isolated polypeptide as described above.

Preferably, the method includes adding at least one additional enzyme useful for improving dough and/or a baked product made therefrom. Preferably, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Preferably, the additional enzyme is an amylase which is preferably an exoamylase. Preferably, the exoamylase is a maltogenic amylase. Preferably, the exoamylase is a non-maltogenic amylase. Preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. Preferably, the phospholipase comprises SEQ ID NO: 5 or an active fragment thereof. Preferably, the active fragment is a mature polypeptide.

Preferably, the method includes adding an emulsifier. Preferably, the emulsifier is a phospholipid emulsifier. Preferably, the phospholipid emulsifier is lecithin or lyso-lecithin. Preferably, the emulsifier is a non-phospholipid emulsifier. Preferably, non-phospholipid emulsifier is DATEM, SSL, a monoglyceride or a diglyceride.

In another aspect of the present invention, a pre-mix for baking is presented having flour and an isolated polypeptide as described above.

Preferably, the pre-mix includes adding at least one additional enzyme useful for improving dough and/or a baked product made therefrom. Preferably, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Preferably, the additional enzyme is an amylase which is preferably an exoamylase. Preferably, the exoamylase is a maltogenic amylase. Preferably, the exoamylase is a non-maltogenic amylase. Preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. Preferably, the phospholipase comprises SEQ ID NO: 5 or an active fragment thereof. Preferably, the active fragment is a mature polypeptide.

In another aspect of the present invention, a method of creating a lyso-galactolipid in a lipid containing food matrix is presented having the step of adding to the lipid containing food matrix an isolated polypeptide as described above. Preferably, the lipid containing food matrix is selected from the group consisting of eggs and food products containing eggs, dough for sweet bakery goods, processed meat, milk based products, and vegetable oil.

In another aspect of the present invention, a method of creating a lyso-galactolipid in a lipid containing animal feed matrix or a grain based matrix is presented having the step of adding to the lipid containing to the lipid containing animal feed matrix or the grain based matrix an isolated polypeptide as described above.

In another aspect of the present invention, a dough is presented having an isolated polypeptide as described above. Preferably, the dough has improved dough extensibility and/or stability. Preferably, the dough has at least one additional enzyme useful for improving dough and/or a baked product made therefrom. Preferably, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Preferably, the additional enzyme is an amylase which is preferably an exoamylase. Preferably, the exoamylase is a maltogenic amylase. Preferably, the exoamylase is a non-maltogenic amylase. Preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. Preferably, the phospholipase comprises SEQ ID NO: 5 or an active fragment thereof. Preferably, the active fragment is a mature polypeptide.

In another aspect of the present invention, a method of preparing a baked product is presented having the step of baking a dough as described above.

In another aspect of the present invention, a baked product is presented prepared according to the method as described above. Preferably, the baked product has at least one improved property selected from the group consisting of improved crumb pore size, improved uniformity of gas bubbles, no separation between crust and crumb, increased volume, increased crust crispiness and improved oven spring.

In another aspect of the present invention, an isolated polypeptide is presented constituting a lipase having at least 75, 80, 85, 90, 95, 98, 99 or 100% sequence identity to SEQ ID NO:3 or SEQ ID NO:4 or a lipase active fragment thereof. Preferably, the lipase active fragment is a mature polypeptide.

### EXAMPLES

### Assays and Methods

### Enzyme Characterization Assays

### PC-U Assay:

Phospholipase activity (PC-U) was determined using the following assay: A substrate solution was made by dissolving 1% (w/v) L-α Phosphatidylcholine Soy-95% (Avanti 441601G, Avanti Polar Lipids, USA), 6.5% (w/v) TRITON™-X 100 (Sigma X-100), and 5 mM CaCl2 in 0.05 M HEPES buffer pH 7.0. Enzyme samples and oleic acid standard (WakoChemicals GmbH, Germany, NEFA Standard, #270-77000) were diluted in 10 mM HEPES pH 7.0 containing 0.1% TRITON™ X-100. Enzyme samples were diluted to an appropriate concentration to achieve a linear response in the assay. Analysis was carried out in a 96 well micro titer plate (ThermoScientific #269620) and using a thermomixer (iEMS Incubator/shaker, Thermo Scientific). The assay was run as follows; 35 μL of substrate solution is mixed with 5 μL of enzyme sample and incubated for 600 sec at 30° C. The amount of free fatty acid liberated during enzymation was measured using the NEFA kit (WakoChemicals GmbH, Germany, NEFA-HR (2) R1 #434-91795 and NEFA-HR (2) R2 #436-91995).

The NEFA kit is composed of two reagents:

### NEFA-HR (2) R1:

50 mM Phosphate buffer pH 7.0 containing
0.53 U/mL Acyl-CoA Synthase (ACS)
0.31 mM coenzyme A (CoA)
4.3 mM adenosine 5-triphosphate disodium salt (ATP)
1.5 mM 4-amino-antipyrine (4-AA)
2.6 U/mL Ascorbate oxidase (AOD)
0.062% Sodium azide

### NEFA-HR (2) R2:

2.4 mM 3-Methyl-N-Ethyl-N-(E-Hydroxyethyl)-Aniline (MEHA)
12 U/mL Acyl-CoA oxidase (ACOD)
14 U/mL Peroxidase (POD)

After incubation the substrate-enzyme mixture is added 113 μL NEFA-HR (2) R1 and incubated for 300 sec at 37° C. Next, 56 μL NEFA-HR (2) R2 was added and the mixture was incubated for 300 sec at 37° C. Finally, OD 540 nm was measured (Molecular Devices, SpectraMaxPlus).

Enzyme activity was calculated based on the use of an enzyme standard of SEQ ID NO: 1. The enzyme standard was assigned an activity of 12174 PC-U/g based on the use of an oleic acid standard. Enzyme activity PC-U is calculated as micromole fatty acid produced per minute under assay conditions.

### LPC-U Assay:

Lysophospholipase activity (LPC-U) was determined using the following assay: A substrate solution was made by dissolving 1% 1-oleoyl-2-hydroxy-sn-Glycero-3-phosphocholine (Avanti 845875P, Avanti Polar lipid, USA), 6.5% (w/v) TRITON™-X 100 (Sigma X-100), and 5 mM CaCl2 in 0.05 M HEPES buffer pH 7.0. Enzyme samples and oleic acid standard (WakoChemicals GmbH, Germany, NEFA Standard, #270-77000) were diluted in 10 mM HEPES pH 7.0 containing 0.1% TRITON™ X-100. Enzyme samples were diluted to an appropriate concentration to achieve a linear response in the assay. Analysis was carried out in a 96 well micro titer plate

ThermoScientific #269620) and using a thermomixer (iEMS Incubator/shaker, Thermo Scientific). The assay was run as follows; 35 μL of substrate solution is mixed with 5 μL of enzyme sample and incubated for 600 sec at 30° C.

The amount of free fatty acid liberated during enzymation was measured using the NEFA kit and enzyme activity was calculated based on the use of an enzyme standard of SEQ ID NO: 1. The enzyme standard was assigned an activity of 364 LPC-U/g based on the use of an oleic acid standard.

### DGDG-U Assay:

Galactolipase activity (DGDG-U) was determined using the following assay: A substrate solution was made by dissolving 1% Di-galactosyl-di-glyceride (with a purity >80%, possibly supplied by Sigma Aldrich or Avanti Polar Lipids), 6.5% (w/v) TRITON™-X 100 (Sigma X-100), and 5 mM CaCl2 in 0.05 M HEPES buffer pH 7.0. Enzyme samples and oleic acid standard (WakoChemicals GmbH, Germany, NEFA Standard, #270-77000) were diluted in 10 mM HEPES pH 7.0 containing 0.1% TRITON™ X-100. Enzyme samples were diluted to an appropriate concentration to achieve a linear response in the assay. Analysis was carried out in a 96 well micro titer plate (ThermoScientific #269620) and using a thermomixer (iEMS Incubator/shaker, Thermo Scientific). The amount of free fatty acid liberated during enzymation was measured using the NEFA kit (WakoChemicals GmbH, Germany, NEFA-HR (2) R1 #434-91795 and NEFA-HR (2) R2 #436-91995).

The amount of free fatty acid liberated during enzymation was measured using the NEFA kit and enzyme activity was calculated based on the use of an enzyme standard of SEQ ID NO: 1. The enzyme standard was assigned an activity of 3228 DGDG-U/g based on the use of an oleic acid standard.

### Protein Determination by Stain Free Imager Criterion

Protein was quantified by SDS-PAGE gel and densitometry using GelDoc™ Go Imaging system (Bio-Rad). Reagents used in the assay: Concentrated (2×) Laemmli Sample Buffer (Bio-Rad, Catalogue #1610737); 26-well TGX Any kDa Gel (Bio-Rad, Catalogue #5678125); protein markers “Precision Plus Protein™ Unstained Protein Standards” (Bio-Rad, Catalogue #161-0363); protein standard based on SEQ ID NO: 1 (protein concentration assigned by Total Amino Acid Analysis, Eurofins Scientific). The analysis was carried out as follow: In a 96 well-PCR plate 50 μL diluted enzyme sample were mixed with 50 μL sample buffer containing 2.7 mg DTT. The plate was sealed by Microseal ‘B’ Film from Bio-Rad and placed into PCR machine and heated at 70° C. for 10 minutes. Next the chamber was filled with running buffer, gel cassette was set. Then 10 μL of each sample and standard (~0.1-1.00 mg/mL SEQ ID NO:1) was loaded on the gel and 10 μL of the markers were loaded. After that the electrophoresis was run at 200 V for 34 min. Following electrophoresis the gel was transferred to the GelDoc Go Imager. Image Lab software was used for calculation of intensity of each band. By knowing the protein amount of the standard sample a calibration curve was created. The amount of target protein in the sample was determined by the band intensity and the calibration curve.

Crusty Roll baking setup

| Recipe | Bakers % |
|---|---|
| Wheat flour (Reform) | 100 |
| Compressed yeast (Malteserkors) | 4.5 |
| Salt | 1.6 |
| Sucrose | 1.6 |
| Water (400 BU-2%) | 59 |
| Fungal alpha amylase (16.2 FAU/g blend) | 0.47 |
| Other Enzymes | variable |

Kneading on a Diosna spiral mixer. Water uptake for flour according to analysis: 400 BU-2%

### Procedure

Mix all ingredients in a bowl, 1 minute slow speed-add water and knead 2 minutes slow and 6.5 minutes fast speed. Dough temperature must be approximate 26° C. 1350 g dough is scaled and molded round by hand. The dough is rested in a heating cabinet for 10 minutes at 30° C.

The dough is molded into 30 dough balls on a “GLIMIK™ rounder”—settings according to table on machine.

The dough is proofed for 45 minutes at 34° C., 85% RH and baked for 13 minutes at 200° C./21 steam+5 minutes damper open (MIWE oven prog. 1). After baking the rolls are cooled for 25 minutes at ambient temperature before weighing and measuring of volume.

Dough and bread characteristics are evaluated by a skilled person.

TABLE 2
Dough and Bread Characteristics and Evaluation

| Evaluation | Evaluation method | Lowest score = 1 | Highest score = 10 |
|---|---|---|---|
| Dough | | | |
| Dough | Extend dough with | Dough cannot be | Dough can be stretched |
| development | fingers | stretched without | obtaining papery thin |
| after mixing | | breaking | dough without breakage |
| Stickiness after | Cut a big slit in all | Dry surface. The | The dough sticks to |
| mixing | dough, open the dough, | dough slips your | your fingers |
| | touch the cut dough | fingers | |
| | surface with fingers | | |
| Extensibility | Extend dough with | Dough cannot be | Dough can be stretched |
| after resting | fingers | stretched without | obtaining papery thin |
| | | breaking | dough without breakage |
| Stickiness after | Cut a big slit in all | Dry surface. The | The dough sticks to |
| resting | dough, open the dough, | dough slips your | your fingers |
| | touch the cut dough | fingers | |
| | surface with fingers | | |
| Crust | | | |
| Crispiness of | Fracture crust using | Leathery crust | Crisp crust |
| crust | several fingers | | |
| Crumb | | | |
| Crumb pore size | Visual evaluation of | Open crumb, big gas | Fine crumb, small gas |
| | sliced bread, | bubbles | bubbles |
| | size of gas bubbles | | |
| | in crumb | | |
| Crumb pore | Visual evaluation of | Big variation in sizes | Constant gas bubble |
| homogeneity | sliced bread, | of gas bubbles | size |
| | homogeneity of gas | | |
| | bubbles | | |
| Product shape | | | |
| Capping/ | Visual evaluation of | A very large hole | No separation between |
| Hole under the | vertical cut surface | directly under the | crust and crumb. |
| crust | | crust. | |
| Oven | Visual evaluation | No energy | High level of energy |
| spring/Energy | amount of energy in the | | |
| | product | | |

### Example 1. Preparation of Lipases

Lipases of the present invention, having galactolipase activity and no and or low lysophospholipase activity can be wild type or engineered. Libraries of lipases can be screened for the appropriate properties according to the present invention. Alternatively, a lipase not having the desired properties can be modified via protein engineering to provide a lipase according to the present invention.

DNA manipulations to provide lipases of the present invention, i.e. lipases having galactolipase activity and no or low lyso-phospholipase activity, may be carried out using molecular biology techniques known in the art. Polynucleotide fragments corresponding to the coding sequences for the various lipases may be synthesized using preferred codons for the fungal expression host Trichoderma reesei (T. reesei) and assembled using PCR techniques. A suitable signal sequence e.g. the pep1 aspartate protease from T. reesei is introduced at the N-terminus (5′ end) of each lipase gene sequence. The Gateway® BP recombination technique is used to introduce the genes into the pDonor221 vector (Invitrogen, US) according to recommendations of the supplier. The resulting entry plasmids may be recombined with the destination vector pTTTpyr2 resulting in final expression vectors. pTTTpyr2 is similar to the pTTTpyrG vector described before (PCT publication WO 2011/063308), except that the pyrG gene is replaced with the pyr2 gene. Vector pTTTpyr2 contains the T. reesei cbhI promoter and terminator regions, the Aspergillus nidulans amdS selection marker, the T. reesei pyr2 selection marker, and telomeric sequences from T. reesei (for autonomous maintenance). These plasmids may be propagated in Escherichia coli TOP10 cells (Invitrogen, US), and the DNA is purified, and sequence verified.

All fungal manipulations, including high throughput transformations, inoculations, fermentations and harvesting may be performed robotically in either 24 or 96 well microtiter plates (MTP). Plasmids may be transformed into suitable T. reesei host strain using the polyethylene glycol (PEG)-protoplast method. In brief, transformation mixtures containing approximately 0.5-2 μg of DNA and 5×106 protoplasts in a total volume of 50 μL may be treated with 200 μL of 25% PEG solution followed by dilution with equal volume of 1.2 M sorbitol/10 mM Tris/10 mM CaCl2 pH 7.5 solution and poured in 24 well MTPs with 1 ml of 3% low melting agarose containing 1M sorbitol in minimal medium. After sufficient growth transformants from each well may be pooled together and plated on fresh 24 well agar MTPs. Once sporulated, spores may be harvested and used for inoculation of liquid cultures.

For the expression of lipase proteins, the transformed T. reesei strains may be cultured as follows: 10⁵-10⁶ T. reesei spores may be used to inoculate 1 ml of production medium (37 g/L glucose, 1.6 g/L sophorose, 9 g/L casamino acids, 10 g/L (NH4) 2SO4, 4.5 g/L KH₂PO4, 1 g/L MgSO4*7H2O, 1 g/L CaCl2*2H2O, 33 g/L PIPPS buffer (pH 5.5), 0.25% T. reesei trace elements (100%: 175 g/L citric acid (anhydrous), 200 g/L FeSO4*7H2O, 16 g/L ZnSO4*7H2O, 3.2 g/L CuSO4*5H2O, 1.4 g/L MnSO4*H2O, 0.8 g/L H3BO3) in 24 well MTPs. MTPs may be incubated in a shaker incubator with a 50 mm throw at 200 rpm and 28 C with 80% humidity. After 5 days of fermentation, the cultures may be filtered by centrifugation using hydrophilic PVDF membranes to obtain clarified supernatants used for analysis of the recombinant lipase enzymes.

### Example 2. Purification and Quantification of Lipases

T. reesei strains encoding lipases of the present invention may be cultured as described above, and clarified supernatants may be used to determine the lipase substrate specificity. The lipases of the present invention may alternatively be further purified using the purification methods know in the art. The following method of purification may be used.

Clarified supernatants containing lipase may be concentrated and ammonium sulfate may be added to a final concentration of 1 M. The solution may be loaded onto a HiPrep™ Phenyl FF column pre-equilibrated with 20 mM NaAc (pH 5.0) supplemented with 1 M ammonium sulfate. The target protein may be eluted from the column with 0.1 M ammonium sulfate. The corresponding fractions may be pooled, concentrated and buffer-exchanged into 20 mM Tris (pH 7.5) (Buffer B) using a VivaFlow 200 ultra-filtration device (Sartorius Stedim). The resulting solution may be applied to a HiLoad™ Q FF 16/10 column pre-equilibrated with Buffer B. The target protein may be eluted from the column with 0.3 M NaCl. Fractions containing the active protein may be pooled, concentrated and buffer-exchanged into 20 mM NaAc (pH 5.0), 150 mM NaCl via the 10K Amicon Ultra filtration devices, and then stored in 40% glycerol at −20° C. until usage. Concentration of purified protein may be calculated by measuring the absorbance at 280 nm.

### Example 3. Activity Characterization of Lipases

Enzyme characterization is done by determination of specific activity using different lipid substrates as per activity methods presented in ‘Assays and Methods’. Powerbake 4080 is a commercial product of IFF. Powerbake 4080 is known to have both galactolipase and phospholipase activity. The active enzyme component of Powerbake 4080 is set forth as SEQ ID NO: 6 from U.S. Pat. No. 8,012,732 hereby incorporated by reference (also set forth herein as SEQ ID NO: 1). Lipopan F is a commercial product of Novozymes. The active enzyme in Lipopan F also known to have galactolipase activity and is in SEQ ID NO: 2 of EP0869167B hereby incorporated by reference (also set forth herein as SEQ ID NO: 2).

Lipases of the present invention may be characterized by their activity on 3 different substrates, PC, LPC and DGDG using the assays mentioned under “Assays and Methods”. The results can be seen in table 4.

TABLE 3
Activity of lipases on PC, LPC, DGDG and MGDG substrates.

| SEQ | DGDG- | LPC-U/DGDG-U | PC-U/DGDG-U |
|---|---|---|---|
| ID | U/mg | ratio | ratio |
| 1 | 482 | 0.113 | 3.77 |
| 2 | 1287 | 0.028 | 1.18 |
| 3 | 23608 | 0.005 | 0.12 |
| 4 | 18092 | 0.011 | 0.04 |

### Example 4. Baking Experiments Testing Application Effect

In this experiment the candidates having low LPC-U/DGDG-U ratio were tested in a Crusty Roll experimental setup to show application performance.

The Crusty Roll baking was done according to ‘Crusty Roll’ description presented in the ‘Assay and Methods’ section above.

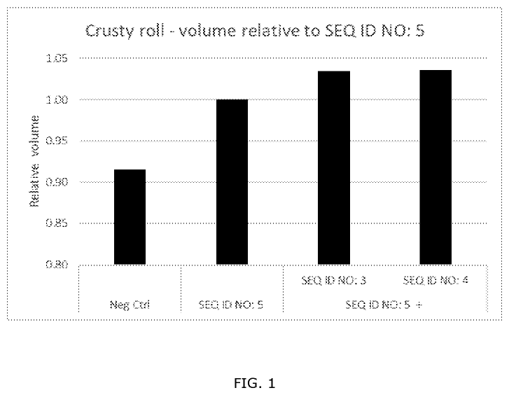

The experimental setup of the application trials and the results from the baking evaluation are presented in respectively Table 4 and FIG. 1.

TABLE 4
Experimental setup evaluating LPC-U/DGDG-U candidates
in Crusty Roll, testing LPC-U/DGDG-U candidate
response baking on top of SEQ ID NO: 5

| | | | | SEQ ID | SEQ ID |
|---|---|---|---|---|---|
| | | | | NO: 5 + | NO: 5 + |
| | | Negative | SEQ ID | SEQ ID | SEQ ID |
| | | Control | NO: 5 | NO: 3 | NO: 4 |
| Dosage | SEQ ID NO: 5, | — | 8800 | 8800 | 8800 |
| U/kg | PC-U/kg flour | | | | |
| flour | LPC-U/DGDG-U | — | — | 100 | 100 |
| | candidate, DGDG- | | | | |
| | U/kg flour | | | | |

What is claimed is:

1. An isolated polypeptide comprising a lipase having galactolipase activity of at least 100 DGDG-U per mg of said lipase and having a ratio of LPC-U/DGDG-U below 0.02.

2. The isolated polypeptide of claim 1 having galactolipase activity of at least 150 DGDG-U per mg.

3. The isolated polypeptide of claim 2 having galactolipase activity of at least 200 DGDG-U per mg.

4. The isolated polypeptide of claim 3 having galactolipase activity of at least 250 DGDG-U per mg.

5. The isolated polypeptide of claim 4 having galactolipase activity of at least 300 DGDG-U per mg.

6. The isolated polypeptide of any of claims 1 to 5 having a ratio of LPC-U/DGDG-U below 0.01.

7. The isolated polypeptide of claim 6 having a ratio of LPC-U/DGDG-U below 0.009.

8. The isolated polypeptide of claim 7 having a ratio of LPC-U/DGDG-U below 0.007.

9. The isolated polypeptide of claim 8 having a ratio of LPC-U/DGDG-U below 0.005.

10. The isolated polypeptide of claim 9 having a ratio of LPC-U/DGDG-U below 0.003.

11. The isolated polypeptide of claim 10 having a ratio of LPC-U/DGDG-U below 0.001.

12. The isolated polypeptide of any of claims 1 to 11 further having a ratio of PC-U/DGDG-U above 0.01.

13. The isolated polypeptide of any of claims 1 to 12 wherein said lipase is an engineered lipase.

14. An isolated polynucleotide comprising a nucleic acid sequence encoding the isolated polypeptide of any one of claims 1 to 13.

15. A recombinant expression vector comprising a polynucleotide according to claim 14.

16. A host cell comprising the recombinant expression vector according to claim 15.

17. A method of making a dough, said method comprising admixing a dough component selected from the group consisting of flour, salt, water, sugar, fat, lecithin, oil and yeast with an isolated polypeptide according to any of claims 1 to 13.

18. The method of making a dough of claim 17 further comprising adding at least one additional enzyme useful for improving dough and/or a baked product made therefrom.

19. The method of making a dough of claim 18 wherein the additional enzyme is an amylase, cyclodextrin glucanotransferase, peptidase, transglutaminase, lipase other than said isolated polypeptide according to any of claims 1 to 13, phospholipase, galactolipase, cellulase, hemicellulase, protease, protein disulfide isomerase, glycosyltransferase, peroxidase, lipoxygenase, laccase, lactase, or oxidase.

20. The method of claim 19 wherein said amylase is an exoamylase.

21. The method of claim 20 wherein said exoamylase is a maltogenic amylase.

22. The method of claim 20 wherein said exoamylase is a non-maltogenic amylase.

23. The method of claim 22 wherein said non-maltogenic amylase hydrolyses starch by cleaving off one or more linear malto-oligosaccharides, predominantly comprising from four to eight D-glucopyranosyl units, from the non-reducing ends of the side chains of amylopectin.

24. The method of claim 19 wherein said additional enzyme is a phospholipase.

25. The method of claim 24 wherein the phospholipase comprises SEQ ID NO: 5 or an active fragment thereof.

26. The method of claim 25 wherein the active fragment is a mature polypeptide.

27. The method of any of claims 17 to 26 further comprising adding an emulsifier.

28. The method of claim 27 wherein the emulsifier is a phospholipid emulsifier.

29. The method of claim 28 wherein the phospholipid emulsifier is lecithin or lyso-lecithin.

30. The method of claim 29 wherein the emulsifier is a non-phospholipid emulsifier.

31. The method of claim 30 wherein the non-phospholipid emulsifier is DATEM, SSL, a monoglyceride or a diglyceride.

32. A pre-mix for baking comprising flour and an isolated polypeptide according to any of claims 1-13.

33. A pre-mix according to claim 32 further comprising at least one additional enzyme useful for improving dough and/or a baked product made therefrom.

34. A pre-mix according to claim 33 wherein the additional enzyme is an amylase, cyclodextrin glucanotransferase, peptidase, transglutaminase, lipase other than said isolated polypeptide according to any of claims 1 to 13, phospholipase, galactolipase, cellulase, hemicellulase, protease, protein disulfide isomerase, glycosyltransferase, peroxidase, lipoxygenase, laccase, lactase, or oxidase.

35. The pre-mix of claim 34 wherein said amylase is an exoamylase.

36. The pre-mix of claim 35 wherein said exoamylase is a maltogenic amylase.

37. The pre-mix of claim 36 wherein said exoamylase is a non-maltogenic amylase.

38. The pre-mix of claim 36 wherein said non-maltogenic amylase hydrolyses starch by cleaving off one or more linear malto-oligosaccharides, predominantly comprising from four to eight D-glucopyranosyl units, from the non-reducing ends of the side chains of amylopectin.

39. The pre-mix of claim 34 wherein said additional enzyme is a phospholipase.

40. The pre-mix of claim 39 wherein the phospholipase comprises SEQ ID NO: 5 or an active fragment thereof.

41. The pre-mix of claim 40 wherein the active fragment is a mature polypeptide.

42. A method of creating a lyso-galactolipid in a lipid containing food matrix comprising adding to the lipid containing food matrix an isolated polypeptide according to any of claims 1 to 13.

43. The method of creating a lyso-galactolipid in a lipid containing food matrix of claim 42 wherein said lipid containing food matrix is selected from the group consisting of eggs and food products containing eggs, dough for sweet bakery goods, processed meat, milk based products, and vegetable oil.

44. A method of creating a lyso-galactolipid in a lipid containing animal feed matrix or a grain based matrix comprising adding to the lipid containing animal feed matrix or the grain based matrix an isolated polypeptide according to any of claims 1 to 13.

45. A dough comprising an isolated polypeptide according to any of claims 1-13.

46. The dough of claim 45 having improved dough extensibility and/or stability.

47. The dough of claim 45 or 46 further comprising at least one additional enzyme useful for improving dough and/or a baked product made therefrom.

48. The dough of claim 47 wherein the additional enzyme is an amylase, cyclodextrin glucanotransferase, peptidase, transglutaminase, lipase other than said isolated polypeptide according to any of claims 1 to 13, phospholipase, galactolipase, cellulase, hemicellulase, protease, protein disulfide isomerase, glycosyltransferase, peroxidase, lipoxygenase, laccase, lactase, or oxidase.

49. The dough of claim 48 wherein the additional enzyme is an amylase.

50. The dough of claim 49 wherein said amylase is an exoamylase.

51. The dough of claim 50 wherein said exoamylase is a maltogenic amylase.

52. The dough of claim 51 wherein said exoamylase is a non-maltogenic amylase.

53. The dough of claim 52 wherein said non-maltogenic amylase hydrolyses starch by cleaving off one or more linear malto-oligosaccharides, predominantly comprising from four to eight D-glucopyranosyl units, from the non-reducing ends of the side chains of amylopectin.

54. The dough of claim 48 wherein said additional enzyme is a phospholipase.

55. The dough of claim 54 wherein the phospholipase comprises SEQ ID NO: 5 or an active fragment thereof.

56. The dough of claim 55 wherein the active fragment is a mature polypeptide.

57. A method of preparing a baked product comprising baking a dough according to any of claims 45 to 56.

58. A baked product according to claim 57.

59. The baked product of claim 58 having at least one improved property selected from the group consisting of improved crumb pore size, improved uniformity of gas bubbles, no separation between crust and crumb, increased volume, increased crust crispiness and improved oven spring.

60. An isolated polypeptide comprising a lipase having at least 75, 80, 85, 90, 95, 98, 99 or 100% sequence identity to SEQ ID NO:3 or SEQ ID NO:4 or a lipase active fragment thereof.

61. The isolated polypeptide of claim 60 wherein the lipase active fragment comprises a mature polypeptide.
